(19) United States
(12) Patent Application Publication
(10) Pub. No.: US 2010/0094562 A1
(43) Pub. Date: Apr. 15, 2010

(54) System, Method and Device for Comprehensive Individualized Genetic Information or Genetic Counseling

(76) Inventor: Mordechai Shohat, Petach Tikva (IL)

Correspondence Address: MARSTELLER & ASSOCIATES, P O BOX 803302, DALLAS, TX, 75380-3302, US

(21) Appl. No.: 12/598,219

(22) PCT Filed: May 4, 2008
(86) PCT No.: PCT/IL08/00602

§ 371 Date: Dec. 27, 2009

Related U.S. Application Data
(60) Provisional application No. 60/924,230, filed on May 4, 2007.

(30) Foreign Application Priority Data
Nov. 6, 2007 (IL) 187204

(51) Int. Cl. G06F 17/18; G06Q 50/00; G06F 19/00

(52) U.S. Cl. 702/19; 705/2; 702/179

(57) ABSTRACT

A method and system for analysis of genetic information. Preferably, such analysis enables genetic counseling to be provided to a patient and/or relative, in which such counseling includes conveying at least one aspect of the analysis in lay terms.

### FIELD OF THE INVENTION

The present invention is of a method and system for genetic counseling, and in particular, of such a method and system for providing a comprehensive, personalized analysis of genetic and/or related information for an individual.

### BACKGROUND OF THE INVENTION

The area of medical genetics is rapidly expanding, requiring physicians to obtain more information about medical conditions for individual patients that may have implications for themselves and their relatives. With such expansion, physicians may now order more and different genetic tests. Determination of which tests should be ordered is increasingly difficult, particularly for primary care physicians or physicians who have specialized in fields such as obstetrics. These physicians may be expected or required to order testing, yet are not expert in this field. Furthermore, they may not be able to explain the tests and their implications to patients in a full and complete manner. Unfortunately, there are not sufficient genetic counselors to provide such information to every patient.

With the amount of genetic information physicians need to convey to their patients, and with the shortage in genetic counselors, computer based programs could be very useful in allowing both shifting of some of the information/counseling of “simple” cases to the primary physician and self education of people prior to a scheduled session for genetic counseling. By educating the patients prior to the formal genetic counseling, the counseling sessions become more efficient and focused on questions that the patients still have after the basic information was given. Furthermore, such programs could help physicians to order the most useful and informative tests, again increasing the efficiency and productivity of genetic counseling sessions. Unfortunately, no such program is available which is aimed at the non-expert in this field.

### SUMMARY OF THE INVENTION

The background art does not teach or suggest a method or system for genetic counseling which is suitable for a non-expert. The background art also does not teach or suggest a method or system for analysis of genetic information, including information from genetic tests.

The present invention overcomes these drawbacks of the background art by providing a method or system for analysis of genetic information. Preferably, such analysis enables genetic counseling to be provided to a patient and/or relative, in which such counseling includes conveying at least one aspect of the analysis in lay terms. By “lay terms” it is meant language which is suitable for a non-genetic expert, and preferably which is suitable for non-medical personnel. By “non-medical personnel” it is meant individuals who are not doctors, nurses, paramedics or others with medical training.

The present invention preferably provides information regarding genetic counseling which is required by patients and/or their relatives, and/or by medical providers, for before or after genetic counseling. Furthermore, according to some embodiments, the present invention preferably suggests one or more genetic tests to be performed according to an analysis of genetic information. Such genetic information may optionally include but is not limited to one or more of a result of a previously performed genetic test, ethnic background of the individual to be tested, one or more medical history factors of the individual to be tested, any current symptoms of the individual to be tested, one or more medical history factors of one or more relatives of the individual to be tested, any current symptoms of any such relative, any environmental exposure information (including without limitation working environment, previous exposure to toxins and the like) which might be expected to affect a potential underlying genetic condition, but is not limited to such factors or information.

The present invention has many useful applications. Without limitation, these applications include assisting genetic services in organizations such as health maintenance organizations and/or any health provider, and/or for assisting the medical staff of any organization which provides genetic counseling.

Implementation of the method and system of the present invention can significantly shorten the time spent by the genetic counselor, and at the to same time improve it by allowing pre-counseling education so that there is less to explain during the counseling session. Furthermore, by providing uniformity in administrative decisions (for example with regard to which tests to order) and also consistent recording and analysis of results, it enables a high standard of medical care to be consistently provided, even through non-genetic experts such as primary care physicians, obstetricians and other medical personnel who are not experts in the field of genetics.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or stages manually, automatically, or a combination thereof. Moreover, according to actual instrumentation and equipment of preferred embodiments of the method and system of the present invention, several selected stages could be implemented by hardware or by software on any operating system of any firmware or a combination thereof. For example, as hardware, selected stages of the invention could be implemented as a chip or a circuit. As software, selected stages of the invention could be implemented as a plurality of software instructions being executed by a computer using any suitable operating system. In any case, selected stages of the method and system of the invention could be described as being performed by a data processor, such as a computing platform for executing a plurality of instructions.

Although the present invention is described with regard to a “computer” on a “computer network”, it should be noted that optionally any device featuring a data processor and/or the ability to execute one or more instructions may be described as a computer, including but not limited to a PC (personal computer), a server, a minicomputer, a cellular telephone, a to smart phone, a PDA (personal data assistant), a pager, TV decoder, game console, digital music player, ATM (machine for dispensing cash), POS credit card terminal (point of sale), electronic cash register. Any two or more of such devices in communication with each other, and/or any computer in communication with any other computer, may optionally comprise a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagram of an exemplary, illustrative method according to some embodiments of the present invention for genetic counseling;

FIG. 2 is a flowchart of an exemplary, illustrative method according to some embodiments of the present invention for analysis of genetic information for Down syndrome as a non-limiting example;

FIG. 3 is a flowchart of an exemplary, illustrative method according to some embodiments of the present invention for analysis of genetic diseases for an individual and/or family; and

FIG. 4 is a schematic block diagram of an exemplary, illustrative system according to some embodiments of the present invention.

### DETAILED DESCRIPTION OF PREFERRED EMBODIMENTS

The present invention is of a method and system for analysis of genetic information. Preferably, such analysis enables genetic counseling to be provided to a patient and/or relative, in which such counseling includes conveying at least one aspect of the analysis in lay terms.

According to preferred embodiments, there is provided a method for analyzing genetic information of an individual which includes obtaining such information, and analyzing the information to determine which genetic test(s) are recommended. More preferably, once the tests have been performed, the method further comprises analyzing the results in order to determine the statistical likelihood of the tested individual to have a medical condition. By “medical condition” it is meant any disease or physiological defect, or combination thereof, as well as any tendency to suffer from such a disease in the future. The term “disease” as used herein includes any chronic or acute medical condition.

Most preferably, one or more additional tests are suggested as necessary to be performed on the individual and/or on one or more relatives of the individual. For example and without intending to be limiting in any way, such an additional test may optionally provide additional diagnostic information and/or may optionally be used in a situation in which a clear likelihood of a diagnostic conclusion has not been established. The additional test may optionally be a non-genetic test but is preferably a genetic test. The additional test may optionally and preferably be suggested once at least one genetic test has been performed on the individual and/or one or more relatives of the individual.

According to some embodiments, a system according to the present invention comprises a database, a patient information input module and an analyzer. The patient information input module preferably receives information from the individual to be tested and/or one or more relatives. Such information may optionally be provided and/or entered by one or more medical personnel, and preferably includes a medical history. More preferably such information includes the result of at least one genetic test.

The database preferably provides a repository of information regarding the genetic information which includes weights for at least a portion of genetic information. For example, the weighting may preferably determine a weight for a result from a particular genetic test and/or an aspect of the individual's medical history, and so forth. The weighting is preferably used in the analysis to determine a statistical likelihood of an individual suffering from a particular medical condition. The database is optionally and more preferably updated as further research is available for example and/or through machine learning, in which previous test results and other medical information is analyzed to permit one or more weights to be adjusted accordingly.

The analyzer preferably uses the weights and/or other information from the database to analyze the medical and genetic information from the individual being tested (and/or from one or more relatives), in order to provide a statistical likelihood of the individual suffering from a medical condition. More preferably, the analyzer also suggests one or more additional diagnostic tests to be performed, which optionally and most preferably includes at least one genetic test.

The analyzer may optionally use one or more statistical models as is known in the art for assessing risk, based upon the different factors and/or a combination thereof and/or a relationship between a plurality of them. For example with regard to the non-limiting example of pregnancy (and the potential for one or more abnormalities of the fetus), the likelihood ratio could optionally be used, as has been implemented for example for Down syndrome (Palomaki G E, Haddow J E. Maternal serum fetoprotein, age and Down syndrome risk. Am J Obstet Gynecol 1987; 156:460-463).

Optionally and more preferably, the system further includes a to diagnostic test module for operating at least one diagnostic test. The diagnostic test is preferably a genetic test.

Optionally and more preferably, the system further includes a notification module for providing a report to a user regarding the results of the analysis from the analyzer. For example, the notification module may optionally and preferably provide the report through the Internet, for example as a mark-up language document, and/or according to any other messaging mechanism. The report preferably contains different language according to whether it is prepared for medical personnel or for a layperson. The report may also optionally and preferably be prepared according to a template, as described in greater detail below.

Although the description contained herein centers around pregnancy, it should be noted that this is for the purposes of illustration only and is not meant to be limiting in any way. With regard to pregnancy, the medical information preferably includes information regarding such risk factors that include but are not limited to a standard prenatal screening test that yields an abnormal result; an amniocentesis yields an unexpected result (such as a chromosomal defect in the unborn baby); either parent or a close relative has an inherited disease or birth defect; either parent already has children with birth defects or genetic disorders; the mother-to-be has had two or more miscarriages or babies that died in infancy; maternal age for chromosomal anomalies; paternal age; ethnic background; and optionally any environmental exposures by either parent, for example to toxins, radioactivity and so forth.

Examples of prenatal screening tests for which the results may optionally be used include but are not limited to the alpha fetoprotein (AFP) test, hCG (human Chorionic Gonadotropin hormone) test, nuchal translucency, first and second trimester biochemical markers (from the maternal serum and/or from amniotic fluid and/or cells), ultrasound soft markers, as well as any other ultrasound findings. Non-limiting examples of other ultrasound findings include nasal ossification and degree thereof (related to risk for Down syndrome), gross physiological abnormalities and so forth.

Ethnic background may optionally be further related to one or more particular diseases associated with a particular ethnic background. For example, couples of African descent are most at risk for having a child with sickle cell anemia; couples of central or eastern European Jewish (Ashekenazi), Cajun, or Irish descent may be carriers of Tay-Sachs disease; and couples of Italian, Greek, or Middle Eastern descent may carry the gene for thalassemia, a red blood cell disorder.

Amniocentesis is recommended once the combined risk factors reach 1 in 386 for one or more birth defects and/or other genetic conditions of the unborn child. This combination weight may be reached, for example, in mothers-to-be who are 35 or older when the baby is born, even in the absence of other risk factors. Chances of having a child with Down syndrome increase with the mother's age: a woman has a 1 in 350 chance of conceiving a child with Down syndrome at age 35, a 1 in 110 chance at age 40, and a 1 in 30 chance at age 45.

The principles and operation of the present invention may be better understood with reference to the drawings and the accompanying description.

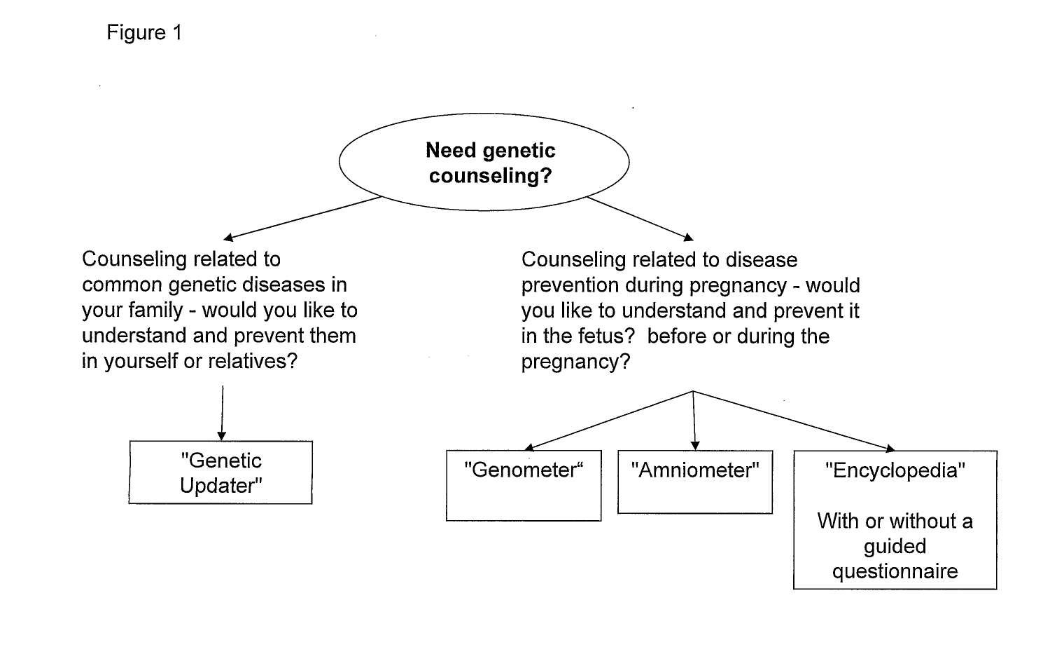

Referring now to the drawings, FIG. 1 is a schematic block diagram of an exemplary, illustrative method according to some embodiments of the present invention for genetic counseling.

As is shown, the method first involves determining whether the individual (and/or a relative of the individual) wishes to be counseled regarding one or more genetic diseases for that individual and/or relative (left branch) or whether counseling is desired regarding pregnancy. The left branch relates to common (or uncommon) genetic diseases existing in the individual and/or the family; it preferably provides one or more of information about the individual risks for specific diseases, recommendations for relevant tests, option for recall and updates of specific subgroups, as well as administrative assistance. This branch and its function are described in greater detail below.

The right branch relates to genetic counseling during pregnancy. Such counseling may optionally be requested by the expectant mother and/or father, and/or may also optionally be requested by the attending physician such as an obstetrician for example. The counseling may optionally be related to the possibility of terminating the pregnancy, and/or may optionally be related to the care to be provided during one or more of the pregnancy, the birth process and/or after the baby is born.

The right branch preferably also provides an explanation of the risks and recommendations for relevant tests as well as administrative assistance.

In more detail, one functional block of the method according to the present invention preferably relates to a genometer, which provides a list of diseases that meet the criteria for carrier screening. More preferably such criteria include but are not limited to the ASHG (American Society of Human Genetics) recommendations, and/or recommendations of any other medical body, and/or the calculated combined ethnic risks of the couple. This functional module is preferably used prior to or at the beginning of a pregnancy.

The expectant mother and/or father preferably are requested to provide information regarding one or more genetic diseases in the family and the ethnic origins of the paternal and the maternal side of the fetus. More preferably, information regarding maternal and (optionally also) paternal age is provided. Also, information regarding genetic testing, previous miscarriages, any birth defects in the parents and/or one or more children of the parents and/or other relative(s), and so forth as described above, is preferably also requested.

The genometer then preferably calculates the risk of each of the couple to be a carrier based on the above information. More preferably at least the carrier frequency in his/her ethnic group(s) is included in the calculation. Other factors preferably include the likelihood that carrier status will be detected in this/these ethnic group(s) and the simplicity of the test required for detection of carrier status in these ethnic groups. Still other factors preferably include one or more other medical information provided by one or both parents, which may optionally indicate a potentially higher personal risk for the fetus. Based on these parameters the program preferably divides the diseases into groups:

Group 1—the disease (and the parents) meet the recommendations for screening.

Group 2—the disease does not meet the criteria for screening, but it is in a gray area and can therefore be offered. Diseases in this category can be further divided into other subgroups, such as diseases that are severe but less common, diseases that are common but mild, etc. A conclusion for such a group may also optionally be further influenced according to one or more recommendations of the attending physician.

Group 3—diseases for which screening is available but not relevant for the couple because of their specific ethnic combination and/or other provided information.

The above functional module may optionally easily be adjusted for any country/population, preferably by adjusting an associated database (see below for a more detailed explanation of an exemplary system according to the present invention).

This functional module is preferably used by OB/GYN physicians and/or other medical personnel before referring couples for genetic screening in countries where screening for carriers of genetic diseases is available.

The second functional module is called the “amniometer” and preferably is used to determine whether amniocentesis is recommended. This module is preferably used during pregnancy, more preferably early in the pregnancy. The module preferably combines information from different tests and/or medical and family history and so forth, for example with regard to the test results and other medical information described above.

The functional module preferably requests the standard and relevant information that affects the risk for conditions for which a test may optionally be performed in amniotic fluid or amniotic cells, including but not limited to maternal (and optionally paternal) age; medical history including but not limited to administration of one or more medicines that affect chromosomal structure or number, for example to the mother to be before she realized that she was pregnant, previous pregnancies with a poor outcome, for example relating to miscarriages, still births and/or infant deaths; the number of fetuses, and if performed, the outcome of fetus reduction and the week at which it was performed; results of tests performed, including but not limited to nuchal translucency, measurement of nasal bone by ultrasound, first and second trimester maternal serum biochemical markers, ultrasound soft markers, as well as any other ultrasound findings.

The functional module then performs an analytic process that preferably combines all the risk results from the various tests (more preferably including at least the risks for Down syndrome and trisomy 18) and calculates the final combined risk for a birth defect, more preferably including at least a risk of Down syndrome.

The third functional module is an “encyclopedia”, which preferably provides a complete genetic questionnaire for a couple with a positive family history or abnormal findings. In addition to an encyclopedia the functional module preferably asks a plurality of questions; more preferably some questions will appear only if the person gives specific answers. The report is preferably comprehensive but written for lay people in lay terms. The report more preferably provides links to obtain additional information that is relevant to their situation. This report saves time during the genetic counseling session and is preferably provided to the expectant mother and/or father before they come to the geneticist.

The report for example optionally and preferably includes an to assessment of relative risk of a syndrome or disease, such as the relative risk of a genetic disease and/or of a condition or disease associated with such a genetic disease. More preferably the risk is also presented in graphic terms, to provide some context regarding the relative risk of such a genetic disease. Optionally and more preferably, one or more further tests that may be recommended are described, optionally including one or more details regarding the test itself, what is tested, the purpose of the test and so forth.

The report may also optionally indicate whether any additional treatment and/or tests and/or counseling would be paid for by a third party, including but not limited to an insurance company, a health maintenance organization, the government or any other third party, or whether the patient and/or a relative would be expected to provide payment. Such an indication is optionally and preferably performed according to any relevant guideline and/or policy of any such organization.

Of course, such a report could optionally be adapted for any type of genetic disease and/or testing, not only for expectant mothers and/or their relatives, for example.

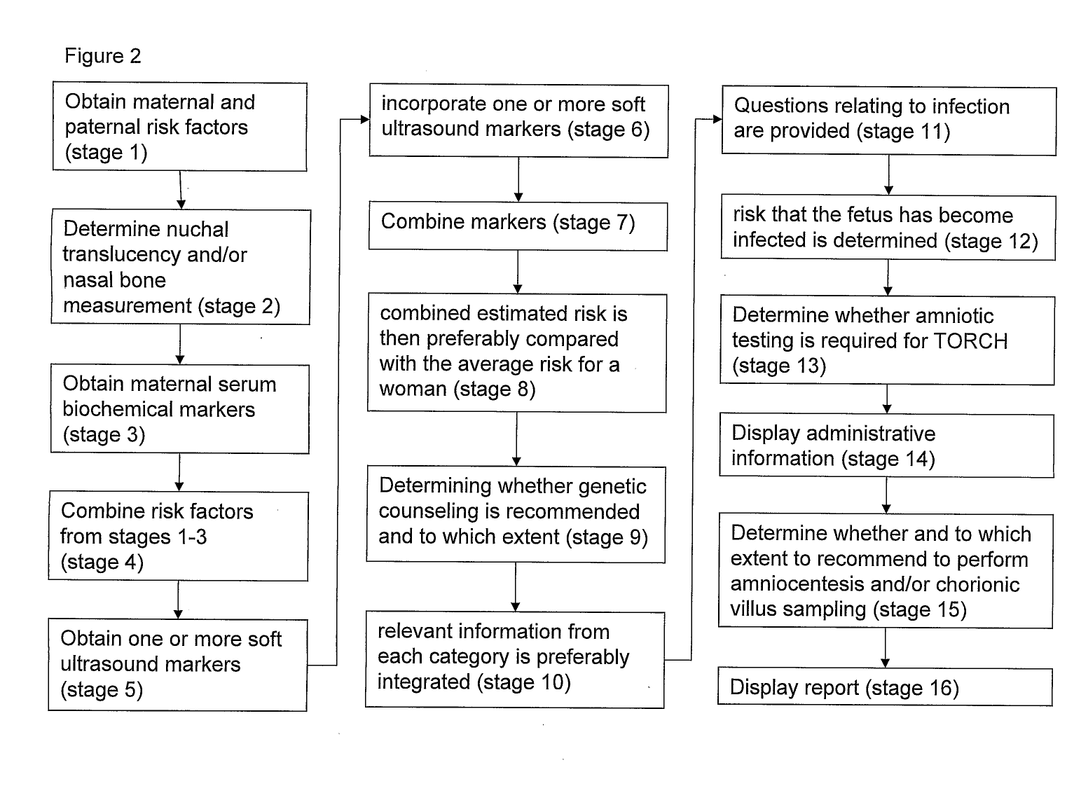

Without wishing to be limited in any way, FIG. 2 relates to an exemplary method for calculating a risk of Down syndrome as one example of an analytical method for genetic counseling according to the present invention.

The calculation in this program of the final combined risk for Down syndrome by the incorporation of the various test results is preferably based on accepted medical recommendations according to the general recommendations of the American Society of Human Genetics and according to the relevant medical literature, as described below.

In stage 1, maternal (and optionally paternal) risk factors are preferably obtained as previously described. In stage 2, nuchal translucency and/or nasal bone measurement is preferably determined. In stage 3, one or more maternal serum biochemical markers are preferably measured. It should be noted that stages 1-3 may optionally be performed in any order; also optionally a stage may be omitted.

In stage 4, the above information is preferably combined. The most accurate estimate to date of the risk for Down syndrome combines the results of the first and second trimester Down syndrome screening tests (nuchal translucency, maternal serum biochemical markers in the first and second trimesters) according to the recommendation of the American Society of Human Genetics.

In stage 5, one or more soft ultrasound markers are preferably collected and in stage 6 the one or more soft ultrasound markers are preferably incorporated to the above calculation. Optionally, such an incorporation may be made at any of stages 1-4; it should also be noted that the order of stages 1-6 is not critical. Again, optionally a stage may be omitted.

The influence of the soft ultrasound markers on the calculated risk for Down syndrome, based on the results of the first and second trimester Down syndrome screening tests, was determined according to the literature. There are currently at least two ways in use for assessing this influence, which were compared on pages 22-23 in vol. 12 of the journal Down Syndrome News (DSNEWS).

The first optional method involves using likelihood ratios for various “soft markers” for Down syndrome as isolated markers with second trimester genetic sonography (Papers by Smith-Bindman JAMA, 2001; Nyberg J Ultrasound Med, 2001; Bromley J Ultrasound Med, 2001; see Reference List at end).

The second optional method involves using likelihood ratios for various “soft markers” for Down syndrome, regardless of whether isolated or multiple, with second trimester genetic sonography (Nicolaides, Ultrasound Obstet Gynecol, 2003; see Reference List at end).

Without wishing to be limited in any way, the first optional method was incorporated for this non-limiting example, with the addition of to modification of the factors to conform to those commonly used by most genetic counselors. Although the original reports indicated that there is a reduction in the risk for Down syndrome if no ultrasound findings have been identified, it was decided (for this non-limiting example) not to adopt this policy in this version as many genetic counselors do not yet do so in common practice. Optionally, such a risk reduction could be incorporated.

Optionally, when the nuchal translucency (NT) measurement is given but there are no data for the NT-based Down syndrome risk, the program calculates the NT-based risk on the assumption that the woman is 11 weeks pregnant at the time the NT was measured (these assumptions provides for a more conservative calculation). Also optionally, the size of the nose is included, particularly with regard to the size of the nasal bone as determined through ultrasound measurements. The nasal bone measurement(s) may optionally be combined with the NT measurement; alternatively, the NT measurement may optionally be used alone. Optionally, additionally or alternatively, one or more other ultrasound soft markers may be combined with the NT measurement and/or nasal bone measurement. Such ultrasound soft markers are described in greater detail below.

The results of the first and second trimester screening tests are then preferably combined in stage 7. The combination of the results of the first and second trimester screening tests is optionally and preferably performed as follows. The program calculates the combined risk once assuming there is no relationship between the two results and once assuming that there is such a relationship. The calculation giving the higher risk is taken as the integrated risk.

Optionally and preferably, any Ministry of Health instructions for amniocentesis are also incorporated to this calculation. These instructions may relate to when such a procedure may be offered and/or when such a procedure is recommended.

In stage 8, the combined estimated risk is then preferably compared with the average risk for a woman as determined according to one or more parameters, and more preferably to the risk of a woman of the same age; optionally the risk is plotted on a schematic graph for display.

In stage 9, it is determined whether genetic counseling is recommended, and also the extent to which it is recommended. If recommended, it is also determined whether a recommendation should be displayed with any associated information.

In stage 10, the relevant information from each category, including offering recommendations for other tests, is preferably integrated. Optionally and preferably other test results, if available, are integrated with the analysis. Such tests may optionally and preferably include ultrasound soft signs, nasal bone measurement and/or nuchal translucency as described herein, as well as any of the other tests described herein, additionally or alternatively.

In stage 11, optionally and preferably, a plurality of questions is provided, more preferably in the form of a specific short questionnaire in which the serological results of the patient for CMV or Toxoplasma (two of the most common intrauterine infections) as well as the serological results of the other TORCH intrauterine infections can be entered. “TORCH” is an acronym standing for Toxoplasmosis, Other Agents, Rubella, Cytomegalovirus, and Herpes Simplex.

In stage 12, optionally and more preferably the risk that the fetus has become infected is determined. This risk is preferably used to determine whether amniotic fluid testing for TORCH is required in stage 13.

In stage 14, preferably administrative information is displayed, more preferably including whether and by whom the procedures are covered in terms of payment and/or insurance. Preferably it is possible to decide which entity pays for the genetic counseling, for example an insurance company, as well as for the amniocentesis and/or chorionic villus sampling if such a procedure is to be performed.

Non-limiting examples of such criteria are given below; such criteria can be easily adjusted to each country or organization based on the local regulations. For example, regarding eligibility for amniocentesis, optionally to the criteria may include one or more of the following factors (but are not limited to such factors): maternal age, preferably being greater than 35 years at the time of the last menstrual period; paternal age; medication which increases the risk for chromosomal anomalies; women younger than 35 years with a final calculated risk for Down syndrome greater than 1:386 at the time of delivery; higher risk determined according to second trimester maternal serum biochemical screening; higher risk determined according to the nuchal translucency measurements in a twin pregnancy; higher risk determined according to the combined and final estimated risk; the parents are both carriers of a Mendelian disease that can be detected prenatally; one of the parents carries a balanced translocation; the couple (or a member thereof, preferably including the mother and/or father) had a previous pregnancy with a documented chromosomal anomaly; or there is a significant ultrasound finding.

The significant ultrasound finding preferably includes any major anomaly, including but not limited to, nuchal translucency greater than 3 mm at 11-13 weeks gestation; and/or more than two ultrasound soft signs present. Such ultrasound soft signs may optionally include but are not limited to one or more of ventriculomegaly (a condition in which the ventricular system of the brain is enlarged); micrognathia; oligohydramnion; two rather than three blood vessels; microcephalus; macrocephalus; hydrocephalus; unilateral ventriculomegaly; borderline ventriculomegaly; cystic hygroma; polyhydramnion (excessive amniotic fluid); echogenic focus in the heart; amniotic band or membrane in the amniotic space; cysts in the neck or in other areas; choroids plexus cyst; edema of the skin, such as the nuchal skin; cardiac defect; malposition of the heart; diaphragmatic hernia; pelvic kidney; hypospadias; spinal abnormalities; hyperechogenic bowel, whether before or after 19 weeks; accumulation of fluid in the pleural space; general edema; dilatation of the renal pelvis and degree of such dilatation; missing kidney; club foot or cleft palate; polydactyly; teratoma; kidney dysplasia or other types of dysplasia; short femur; neural tube defects; omphalocele or gastroschisis; small stomach; cysts in the kidneys; growth retardation; presence of 11 ribs; and/or cortical thumbs. Optionally one or more other such ultrasound soft signs may also be incorporated, in addition or in place of the above list.

The above factors may also optionally and preferably be used for determination of risk, more preferably with regard to stage 10.

In stage 15, it is determined whether amniocentesis and/or chorionic villus sampling are recommended and if so, to what extent, preferably based upon an analysis of at least some and more preferably all of the above criteria.

As previously described with regard to the genometer, optionally and preferably changes may be made to associated data and/or questions to be asked, such as an associated database, without requiring software changes.

In stage 16, a report is preferably provided to the user, who may (for example) be a layperson such as the pregnant woman or a relative, and/or may be medical personnel. The report preferably indicates the level of risk for any particular syndrome and/or genetic condition, and more preferably indicates whether one or more additional tests should be performed. The report may optionally be displayed on any type of display, including but not limited to printing on hard copy, sending a message such as an e-mail message for example, displaying on a monitor or other type of display, and the like.

Optionally, after the above stages are performed at least once, they may optionally be repeated from stage 1, for example after one or more additional test results are obtained and/or one or more tests are repeated. Not all of the stages are necessarily repeated.

Turning now to the functional module relating to genetic counseling for genetic diseases existing in an individual and/or a family, this module preferably provides one or more of information about the individual risks for specific diseases, recommendations for relevant tests, option for recall and updates of specific subgroups, as well as administrative assistance.

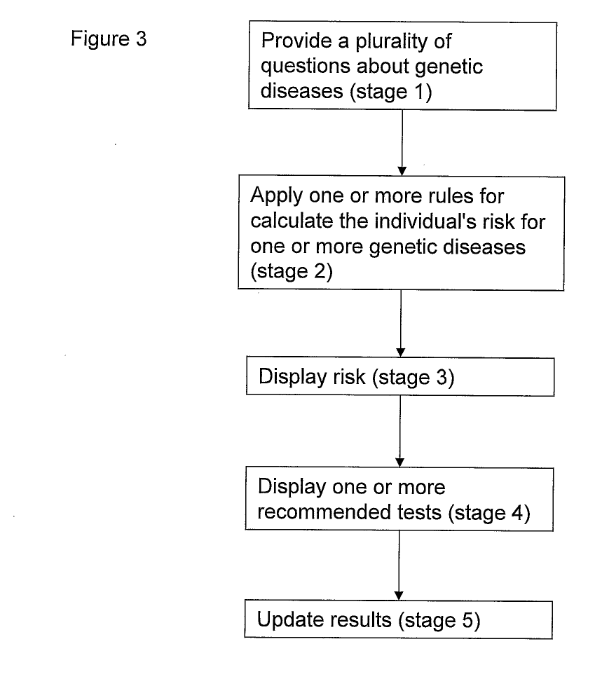

This module is preferably in communication with a genetic updater, which preferably provides at least a plurality of questions and more preferably a complete genetic questionnaire for individuals who are interested in genetic diseases and/or predispositions, most preferably relating to preventing adult onset diseases, as shown in stage 1 of FIG. 3. These diseases may optionally be common in their family and/or in their ethnic group. Preferably the plurality of questions relates to one or more of weight, height, smoking and other habits that can affect predisposition to common adult diseases; medical history; common adult diseases in relatives, more preferably including information about the type of disease, the degree of relation, optionally whether genetic tests have been performed in the affected relative(s) (and more preferably also in the unaffected relatives), and age of onset of these conditions in each of the affected relatives; ethnic origin of each parent; and total number of close relatives on both sides of the family (mother and father).

In stage 2, one or more rules are preferably applied in order to calculate the individual's risk for one or more genetic diseases and more preferably related to the risk for each of the common adult genetic diseases.

Such rules are optionally and preferably applied as follows. The results of the performance of one or more genetic tests are preferably used as the baseline, more preferably including genetic test results for one or more relatives; if these one or more tests have been performed and a mutation has been identified, the test result(s) preferably determine the risk, more preferably according to the standard genetic rules based on which members of the family do or do not carry the mutation among those who have been tested.

If no tests have been performed, then the calculation for each disease is preferably performed according to the contribution of one or more of medical history, age, habits, number of affected relatives for any given disease (more preferably including the degree of relation), and any additional risk compared to the occurrence of the specific disease by chance given the size of the family on the side of the affected relative.

Following estimation of the risk, the risk is preferably displayed, for example to the individual and/or to medical personnel, in stage 3.

Optionally and more preferably it is determined whether any additional tests may be performed, for example most preferably to identify extra risks and/or to determine more accurately whether the individual is at high risk. Such one or more recommended tests are preferably displayed in stage 4.

Such results may optionally be updated as the results become available such that the output is preferably adjusted accordingly, as shown in stage 5. Also preferably information regarding any relevant disease is preferably displayed to the individual and/or medical personnel.

The data may optionally be completed by the individual and/or by medical personnel such as the primary care physician. Preferably it is possible to update the rules and/or to send updates to only those individuals who need updates/recall when these are of practical interest. The messages can then be seen only by the relevant individuals and those people to whom they grant access.

Such updates may optionally be sent to individuals, directly and/or through medical personnel, who correspond to one or more specific medical, ethnic and demographic criteria, about appropriate new tests and/or therapies that are relevant. Such updates may optionally sent through any mechanism as is known in the art, including but not limited to email messages, facsimile, instant messaging (IM), telephone voice communication, SMS (short message service) and/or other types of cellular telephone communication, and so forth.

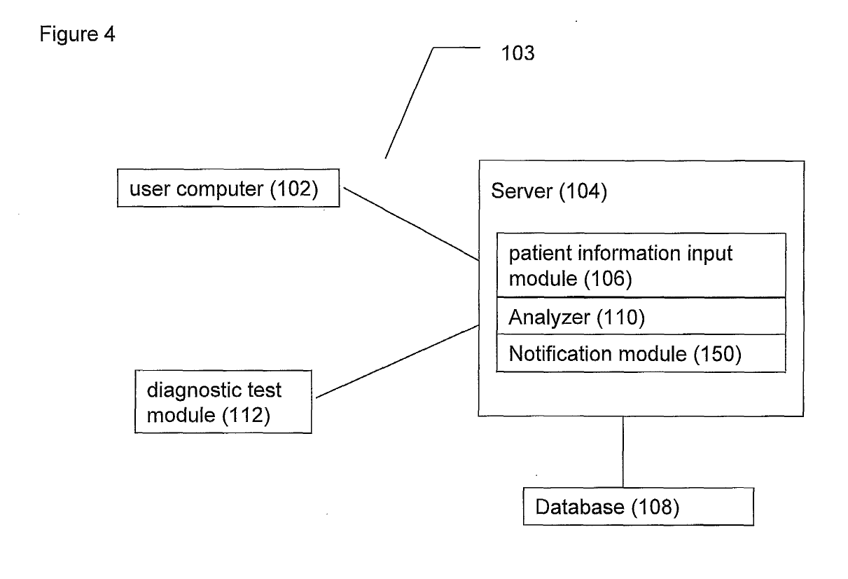

FIG. 4 is a schematic block diagram of an exemplary, illustrative system according to some embodiments of the present invention. A system 100 according to some embodiments of the present invention preferably features a user computer 102 for interacting with a server 104, for example optionally and preferably through a network 103 as shown, which may optionally be the Internet, an intranet or any other computer network (or any type of electronic device network, such as a cellular telephone network) for example. A patient information input module 106 is optionally and preferably operated by server 104 as shown, although alternatively patient information input module 106 is optionally operated by user computer 102. For the former type of operation, patient information input module 106 is optionally and preferably operated through an HTTP (hyper text transfer protocol) server for serving one or more documents according to a mark-up language protocol. Also the former type of operation permits direct updates through server 104, optionally for a plurality of different user computers 102, rather than requiring updates at each user computer 102 separately. Patient information input module 106 preferably provides one or more questions and/or other interface, and then gathers information from the patient and/or a relative and/or medical personnel. In any case, a user interface is preferably provided to a user for entering such information, for example through patient information input module 106 although alternatively a separate interface, such as a web based interface for example, may optionally be provided.

A database 108 preferably includes information relating to one or more medical conditions, more preferably related to genetic diseases, and most preferably the relationship between genetic information and a risk of one or more such diseases. Database 108 also optionally and preferably includes information about one or more users, for example relating to an address (including but not limited to physical, email, instant messaging and the like), telephone number or other contact information for sending a report as described in greater detail below. The information also preferably includes whether the user is medical personnel or is a lay person, and the relationship of the person to the pregnant woman (for example, including but not limited to, medical personnel providing care, another type of caregiver, the pregnant woman herself, the father of the fetus, another relative and so forth). Database 108 may optionally be incorporated within server 104 or may alternatively communicate with server 104 through network 103 for example.

An analyzer 110 preferably combines information from database 108 and information input through patient information input module 106, more preferably to determine risk of the patient and most preferably to recommend one or more additional diagnostic tests.

A diagnostic test module 112 preferably performs the one or more additional diagnostic tests which more preferably include one or more genetic tests. Diagnostic test module 112 may optionally only be an interface for receiving the results of one or more genetic tests, for example if the tests are performed at a laboratory. More preferably, diagnostic test module 112 is integrated with one or more machines and/or computers for performing these tests. In any case, diagnostic test module 112 preferably is able to convert any test results to a uniform format for being analyzed by analyzer 110. Also, diagnostic test module 112 optionally and preferably is able to provide an interface to enable medical personnel to order one or more additional diagnostic tests, such as one or more genetic or other tests, for example. Diagnostic test module 112 optionally and preferably communicates with server 104 through network 103.

Optionally and preferably, server 104 features a notification module 150, for performing one or more notifications. For example, notification module 150 optionally and preferably notifies medical personnel, such as a doctor for example, regarding the outcome of the analysis. Notification module 150 optionally and preferably prepares a report, regarding the outcome of the analysis, and then provides the report to a user, for example through user computer 102. The report may optionally be displayed to the user and/or sent by email or any other messaging mechanism (including but not limited to instant messenger, SMS (short message service) messages, facsimile and so forth). If the user of user computer 102 is not medical personnel, then optionally and preferably the report is both provided to the user and also is sent separately to the appropriate medical personnel, for example optionally through one or more of the above mechanisms. Optionally, additionally or alternatively, if the user of user computer 102 is medical personnel, then the subject of the analysis (for example the expectant mother and/or a relative) may receive the report as well.

Optionally, the report received by medical personnel is different from the report received by the lay person (such as the expectant mother and/or a relative for example); for example, the report received by the former may optionally feature all of the medical terminology, while the report received by the latter may optionally feature lay person's language. These different types of reports may optionally and preferably be accommodated by one or more predetermined templates, which may optionally be complete templates for the report or alternatively partial templates which are then assembled to form the complete report. The template for the lay person would feature lay language while that for the medical personnel would feature more technical, medical professional language.

The report itself may optionally be provided in any type of document format, such as a Word document for example, or any type of mark-up language document, such as an HTML page for example. In any case, if the templates are partial, then they are preferably assembled to form complete documents as is known in the art, for example more preferably according to an overall template “master”, which determines which templates are to be selected (more preferably according to results from analyzer 110) and the order in which the selected templates are to be assembled, which optionally and most preferably is dependent upon the templates that are selected. For example, a partial template may optionally appear in a different section of the report according to one or more other selected partial templates.

### REFERENCES

Bromley B, Lieberman E, Shipp T D, Benacerraf B R. The genetic sonogram: a method of risk assessment for Down syndrome in the second trimester. J Ultrasound Med 2002; 21:1087-1096.
Nicolaides K H. Screening for chromosomal defects. Ultrasound Obstet Gynecol 2003; 21:313-321.
Nyberg D A, Souter V L. Sonographic markers of fetal trisomies: second trimester. J Ultrasound Med 2001; 20:655-674.
Nyberg D A, Souter V L El-Bastawissi A Young S Luthhardt F Luthy D A. Isolated sonographic markers for detection of fetal Down syndrome in the second trimester of pregnancy. J Ultrasound Med 2001; 20:1053-1063.
Smith-Bindman R Hosmer W Feldstein V A Deeks J J, Goldberg J D. Second-trimester ultrasound to detect fetuses with Down syndrome: a meta-analysis. JAMA 2001; 285:1044-1055.

While the invention has been described with respect to a limited number of embodiments, it will be appreciated that many variations, modifications and other applications of the invention may be made.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. A method for analyzing genetic information from an individual comprising:
Receiving genetic information;
Analyzing the genetic information; and
Providing genetic counseling according to the analyzed genetic information.

21. (canceled)

22. (canceled)

23. A system for analyzing genetic information from an individual comprising:
A patient information input module for receiving genetic information regarding the individual and/or a relative, wherein said genetic information comprises test results;
A database for storing a relationship between genetic information and one or more genetic diseases; and
An analyzer for analyzing said genetic information.

24. The system of claim 23, wherein the analyzer analyzes the genetic information according to a weighting system.

25. (canceled)

26. The system of claim 23, wherein the analyzer determines a risk of a genetic disease according to a statistical model incorporating the weighted information.

27. (canceled)

28. (canceled)

29. (canceled)

30. (canceled)

31. (canceled)

32. (canceled)

33. (canceled)

34. (canceled)

35. (canceled)

36. The system of any of claim 23, further comprising a notification module for preparing a report.

37. (canceled)

38. (canceled)

39. The method of claim 20, wherein the genetic counseling includes information relating to a risk of birth defect or other problem with the fetus and/or the pregnancy.

40. A method according to claim 20, further comprising recommending one or more diagnostic test(s).

41. The method of claim 40, wherein the one or more diagnostic tests comprises a genetic test.

42. The method of claim 40, wherein the at least one diagnostic test comprises a biomarker test.

43. The method of claim 20, wherein the individual is pregnant, the method further comprising recommending amniocentesis and/or chorionic villi sampling.

44. The method of claim 20, wherein the genetic information further comprises information regarding one or more risk factors of the individual and/or of a relative.

45. The method of claim 44, wherein the one of more risk factors are determined from a medical history.

46. The method of claim 45, wherein the one or more risk factors are determined from diagnostic information other than a genetic test.

47. The method of claim 45, wherein the one or more risk factors are determined from diagnostic information related to a chronic disease or condition.

48. The method of claim 45, wherein the one or more risk factors are determined from diagnostic information related to a physiological abnormality.
